(19) United States
(12) Patent Application Publication
(10) Pub. No.: US 2017/0014185 A1
(43) Pub. Date: Jan. 19, 2017

(54) TRIAXIAL ANTENNA FOR MICROWAVE TISSUE ABLATION

(71) Applicants: Fred T. Lee, JR., Madison, WI (US); Christopher L. Brace, Middleton, WI (US); Paul F. Laeseke, Madison, WI (US); Daniel Warren van der Weide, Verona, WI (US); Deepak Gopal, Madison, WI (US); Patrick Pfau, Madison, WI (US); Lisa A. Sampson, Cambria, WI (US)

(72) Inventors: Fred T. Lee, JR., Madison, WI (US); Christopher L. Brace, Middleton, WI (US); Paul F. Laeseke, Madison, WI (US); Daniel Warren van der Weide, Verona, WI (US); Deepak Gopal, Madison, WI (US); Patrick Pfau, Madison, WI (US); Lisa A. Sampson, Cambria, WI (US)

(73) Assignee: NeuWave Medical, Inc., Madison, WI (US)

(21) Appl. No.: 15/211,161

(22) Filed: Jul. 15, 2016

Related U.S. Application Data
(63) Continuation of application No. 13/310,022, filed on Dec. 2, 2011, now abandoned.
Application No. 13/310,022 is a continuation-in-part of application No. 13/153,974, filed on Jun. 6, 2011, now abandoned.
Application No. 13/153,974 is a continuation of application No. 11/440,331, filed on May 24, 2006, now abandoned.
Application No. 11/440,331 is a continuation-in-part of application No. 10/834,802, filed on Apr. 29, 2004, now Pat. No. 7,101,369.
Application No. 10/834,802 is a continuation-in-part of application No. 11/237,136, filed on Sep. 28, 2005, now Pat. No. 7,467,015.
Application No. 11/237,136 is a continuation-in-part of application No. 10/834,802, filed on Apr. 29, 2004, now Pat. No. 7,101,369.
Application No. 13/153,974 is a continuation-in-part of application No. 11/236,985, filed on Sep. 28, 2005, now Pat. No. 7,244,254.
Application No. 11/236,985 is a continuation-in-part of application No. 10/834,802, filed on Apr. 29, 2004, now Pat. No. 7,101,369.
Application No. 13/153,974 is a continuation-in-part of application No. 11/237,430, filed on Sep. 28, 2005, now abandoned.
Application No. 11/237,430 is a continuation-in-part of application No. 10/834,802, filed on Apr. 29, 2004, now Pat. No. 7,101,369.
Application No. 10/834,802 is a continuation-in-part of application No. 13/154,934, filed on Jun. 7, 2011, now abandoned.
Application No. 13/154,934 is a continuation of application No. 11/509,123, filed on Aug. 24, 2006, now abandoned.
Application No. 11/509,123 is a continuation-in-part of application No. 11/502,783, filed on Aug. 11, 2006, now abandoned.
Application No. 11/502,783 is a continuation-in-part of application No. 10/834,802, filed on Apr. 29, 2004, now Pat. No. 7,101,369.
Application No. 10/834,802 is a continuation-in-part of application No. 11/452,637, filed on Jun. 14, 2006.
Application No. 11/452,637 is a continuation-in-part of application No. 10/834,802, filed on Apr. 29, 2004, now Pat. No. 7,101,369.
Application No. 11/509,123 is a continuation-in-part of application No. 11/440,331, filed on May 24, 2006, now abandoned.
Application No. 11/440,331 is a continuation-in-part of application No. 10/834,802, filed on Apr. 29, 2004, now Pat. No. 7,101,369.
Application No. 11/509,123 is a continuation-in-part of application No. 11/237,136, filed on Sep. 28, 2005, now Pat. No. 7,467,015.
Application No. 11/237,136 is a continuation-in-part of application No. 10/834,802, filed on Apr. 29, 2004, now Pat. No. 7,101,369.
Application No. 11/509,123 is a continuation-in-part of application No. 11/236,985, filed on Sep. 28, 2005, now Pat. No. 7,244,254.
Application No. 11/236,985 is a continuation-in-part of application No. 10/834,802, filed on Apr. 29, 2004, now Pat. No. 7,101,369.
Application No. 11/509,123 is a continuation-in-part of application No. 11/237,430, filed on Sep. 28, 2005, now abandoned.
Application No. 11/237,430 is a continuation-in-part of application No. 10/834,802, filed on Apr. 29, 2004, now Pat. No. 7,101,369.
Application No. 10/834,802 is a continuation-in-part of application No. 11/502,783, filed on Aug. 11, 2006, now abandoned.
Application No. 11/502,783 is a continuation-in-part of application No. 10/834,802, filed on Apr. 29, 2004, now Pat. No. 7,101,369.
Application No. 10/834,802 is a continuation-in-part of application No. 11/237,136, filed on Sep. 28, 2005, now Pat. No. 7,467,015.
Application No. 11/237,136 is a continuation-in-part of application No. 10/834,802, filed on Apr. 29, 2004, now Pat. No. 7,101,369.
Application No. 11/502,783 is a continuation-in-part of application No. 11/237,430, filed on Sep. 28, 2005, now abandoned.
Application No. 11/237,430 is a continuation-in-part of application No. 10/834,802, filed on Apr. 29, 2004, now Pat. No. 7,101,369.
Application No. 11/502,783 is a continuation-in-part of application No. 11/236,985, filed on Sep. 28, 2005, now Pat. No. 7,244,254.
Application No. 11/236,985 is a continuation-in-part of application No. 10/834,802, filed on Apr. 29, 2004, now Pat. No. 7,101,369.
Application No. 11/502,783 is a continuation-in-part of application No. 11/440,331, filed on May 24, 2006, now abandoned.
Application No. 11/440,331 is a continuation-in-part of application No. 10/834,802, filed on Apr. 29, 2004, now Pat. No. 7,101,369.
Application No. 11/502,783 is a continuation-in-part of application No. 11/452,637, filed on Jun. 14, 2006.
Application No. 11/452,637 is a continuation-in-part of application No. 10/834,802, filed on Apr. 29, 2004, now Pat. No. 7,101,369.
Application No. 10/834,802 is a continuation-in-part of application No. 11/452,637, filed on Jun. 14, 2006.
Application No. 11/452,637 is a continuation-in-part of application No. 10/834,802, filed on Apr. 29, 2004, now Pat. No. 7,101,369.
Application No. 10/834,802 is a continuation-in-part of application No. 11/237,136, filed on Sep. 28, 2005, now Pat. No. 7,467,015.
Application No. 11/237,136 is a continuation-in-part of application No. 10/834,802, filed on Apr. 29, 2004, now Pat. No. 7,101,369.
Application No. 11/452,637 is a continuation-in-part of application No. 11/237,430, filed on Sep. 28, 2005, now abandoned.
Application No. 11/237,430 is a continuation-in-part of application No. 10/834,802, filed on Apr. 29, 2004, now Pat. No. 7,101,369.
Application No. 11/452,637 is a continuation-in-part of application No. 11/236,985, filed on Sep. 28, 2005, now Pat. No. 7,244,254.
Application No. 11/236,985 is a continuation-in-part of application No. 10/834,802, filed on Apr. 29, 2004, now Pat. No. 7,101,369.
Application No. 11/452,637 is a continuation-in-part of application No. 11/440,331, filed on May 24, 2006, now abandoned.
Application No. 11/440,331 is a continuation-in-part of application No. 10/834,802, filed on Apr. 29, 2004, now Pat. No. 7,101,369.
(60) Provisional application No. 60/684,065, filed on May 24, 2005.
(60) Provisional application No. 60/690,370, filed on Jun. 14, 2005.
(60) Provisional application No. 60/702,393, filed on Jul. 25, 2005.
(60) Provisional application No. 60/707,797, filed on Aug. 12, 2005.
(60) Provisional application No. 60/710,276, filed on Aug. 22, 2005.
(60) Provisional application No. 60/710,815, filed on Aug. 24, 2005.
(60) Provisional application No. 60/710,815, filed on Aug. 24, 2005.
(60) Provisional application No. 60/679,722, filed on May 10, 2005.
(60) Provisional application No. 60/684,065, filed on May 24, 2005.
(60) Provisional application No. 60/690,370, filed on Jun. 14, 2005.
(60) Provisional application No. 60/707,797, filed on Aug. 12, 2005.
(60) Provisional application No. 60/702,393, filed on Jul. 25, 2005.
(60) Provisional application No. 60/710,276, filed on Aug. 22, 2005.
(60) Provisional application No. 60/710,815, filed on Aug. 24, 2005.
(60) Provisional application No. 60/710,276, filed on Aug. 22, 2005.
(60) Provisional application No. 60/710,815, filed on Aug. 24, 2005.

(51) Int. Cl. A61B 18/18

(52) U.S. Cl. CPC A61B 18/1815; A61B 2018/00023

(57) ABSTRACT

An improved antenna for microwave ablation uses a triaxial design which reduces reflected energy allowing higher power ablation and/or a smaller diameter feeder line to the antenna.

### CROSS-REFERENCE TO RELATED APPLICATION

This application is

1) a Continuation of U.S. patent application Ser. No. 13/310,022, filed Dec. 2, 2011, which is a Continuation-in-part of U.S. patent application Ser. No. 13/153,974, filed Aug. 30, 2011, which A) is a Continuation of abandoned U.S. patent application Ser. No. 11/440,331, filed May 24, 2006, which is a Continuation-in-Part of pending U.S. patent application Ser. No. 10/834,802, filed Apr. 29, 2004 (now U.S. Pat. No. 7,101,369, which issued Sep. 5, 2006), B) claims priority to expired U.S. Provisional Patent Application No. 60/684,065, filed May 24, 2005, and to expired U.S. Provisional Patent Application No. 60/690,370, filed Jun. 14, 2005, and to expired U.S. Provisional Patent Application No. 60/702,393, filed Jul. 25, 2005, and to expired U.S. Provisional Patent Application No. 60/707,797, filed Aug. 12, 2005, and to expired U.S. Provisional Patent Application No. 60/710,276, filed Aug. 22, 2005, and to expired U.S. Provisional Patent Application No. 60/710,815, filed Aug. 24, 2005, C) is a Continuation-in-Part of pending U.S. patent application Ser. No. 11/237,136, filed Sep. 28, 2005 which issued on Dec. 16, 2008 as U.S. Pat. No. 7,467,015, and which is a continuation-in-part of U.S. patent application Ser. No. 10/834,802, filed Apr. 29, 2004 which issued on Sep. 5, 2006 as U.S. Pat. No. 7,101,369, D) is a Continuation-in-Part of U.S. patent application Ser. No. 11/236,985, filed Sep. 28, 2005, which issued on Jul. 17, 2007 as U.S. Pat. No. 7,244,254, and which is a continuation-in-part of U.S. patent application Ser. No. 10/834,802, filed Apr. 29, 2004 which issued on Sep. 5, 2006 as U.S. Pat. No. 7,101,369, and E) is a Continuation-in-Part of abandoned U.S. patent application Ser. No. 11/237,430, filed Sep. 28, 2005, and which is a continuation-in-part of U.S. patent application Ser. No. 10/834,802, filed Apr. 29, 2004 which issued on Sep. 5, 2006 as U.S. Pat. No. 7,101,369, the contents of each are incorporated herein by reference in their entireties

2) a Continuation-in-part of U.S. patent application Ser. No. 13/154,934, filed Jun. 7, 2011, which is a Continuation of abandoned U.S. patent application Ser. No. 11/509,123, filed Aug. 24, 2006, which A) is a Continuation-in-Part of pending U.S. patent application Ser. No. 11/502,783, filed Aug. 11, 2006, which is a continuation-in-part of U.S. patent application Ser. No. 10/834,802, filed Apr. 29, 2004 which issued on Sep. 5, 2006 as U.S. Pat. No. 7,101,369, B) is a Continuation-in-Part of pending U.S. patent application Ser. No. 11/452,637, filed Jun. 14, 2006, which is a continuation-in-part of U.S. patent application Ser. No. 10/834,802, filed Apr. 29, 2004 which issued on Sep. 5, 2006 as U.S. Pat. No. 7,101,369, C) is a Continuation-in-Part of abandoned U.S. patent application Ser. No. 11/440,331, filed May 24, 2006, which is a continuation-in-part of U.S. patent application Ser. No. 10/834,802, filed Apr. 29, 2004 which issued on Sep. 5, 2006 as U.S. Pat. No. 7,101,369, D) is a Continuation-in-Part of pending U.S. patent application Ser. No. 11/237,136, filed Sep. 28, 2005 which issued on Dec. 16, 2008 as U.S. Pat. No. 7,467,015, and which is a continuation-in-part of U.S. patent application Ser. No. 10/834,802, filed Apr. 29, 2004 which issued on Sep. 5, 2006 as U.S. Pat. No. 7,101,369, E) is a Continuation-in-Part of pending U.S. patent application Ser. No. 11/236,985, filed Sep. 28, 2005, which issued on Jul. 17, 2007 as U.S. Pat. No. 7,244,254, and which is a continuation-in-part of U.S. patent application Ser. No. 10/834,802, filed Apr. 29, 2004 which issued on Sep. 5, 2006 as U.S. Pat. No. 7,101,369, F) is a Continuation-in-Part of abandoned U.S. patent application Ser. No. 11/237,430, filed Sep. 28, 2005, which is a continuation-in-part of U.S. patent application Ser. No. 10/834,802, filed Apr. 29, 2004 which issued on Sep. 5, 2006 as U.S. Pat. No. 7,101,369, G) claims priority to expired U.S. Provisional Patent Application No. 60/710,815, filed Aug. 24, 2005, H) claims priority to expired U.S. Provisional Patent Application No. 60/679,722, filed May 10, 2005, I) claims priority to expired U.S. Provisional Patent Application No. 60/684,065, filed May 24, 2005, J) claims priority to expired U.S. Provisional Patent Application No. 60/690,370, filed Jun. 15, 2005, K) claims priority to expired U.S. Provisional Patent Application No. 60/707,797, filed Aug. 12, 2005, L) claims priority to expired U.S. Provisional Patent Application No. 60/702,393, filed Jul. 25, 2005, and M) claims priority to expired U.S. Provisional Patent Application No. 60/710,276, filed Aug. 22, 2005, the contents of which are incorporated by reference in their entireties;

3) a Continuation-in-part of U.S. patent application Ser. No. 11/502,783, filed Aug. 11, 2006, which A) is a Continuation-in-Part of pending U.S. patent application Ser. No. 10/834,802, filed Apr. 29, 2004 (now U.S. Pat. No. 7,101,369, which issued Sep. 5, 2006), B) is a Continuation-in-Part of pending U.S. patent application Ser. No. 11/237,136, filed Sep. 28, 2005 which issued on Dec. 16, 2008 as U.S. Pat. No. 7,467,015, and which is a continuation-in-part of U.S. patent application Ser. No. 10/834,802, filed Apr. 29, 2004 which issued on Sep. 5, 2006 as U.S. Pat. No. 7,101,369, C) is a Continuation-in-Part of abandoned U.S. patent application Ser. No. 11/237,430, filed Sep. 28, 2005, and which is a continuation-in-part of U.S. patent application Ser. No. 10/834,802, filed Apr. 29, 2004 which issued on Sep. 5, 2006 as U.S. Pat. No. 7,101,369, D) is a Continuation-in-Part of U.S. patent application Ser. No. 11/236,985, filed Sep. 28, 2005, which issued on Jul. 17, 2007 as U.S. Pat. No. 7,244,254, and which is a continuation-in-part of U.S. patent application Ser. No. 10/834,802, filed Apr. 29, 2004 which issued on Sep. 5, 2006 as U.S. Pat. No. 7,101,369, E) is a Continuation-in-Part of abandoned U.S. patent application Ser. No. 11/440,331, filed May 24, 2006, which is a continuation-in-part of U.S. patent application Ser. No. 10/834,802, filed Apr. 29, 2004 which issued on Sep. 5, 2006 as U.S. Pat. No. 7,101,369, F) is a Continuation-in-Part of pending U.S. patent application Ser. No. 11/452,637, filed Jun. 14, 2006, which is a continuation-in-part of U.S. patent application Ser. No. 10/834,802, filed Apr. 29, 2004 which issued on Sep. 5, 2006 as U.S. Pat. No. 7,101,369, G) claims priority to expired U.S. Provisional Patent Application No. 60/710,815, filed Aug. 24, 2005, H)) claims priority to expired U.S. Provisional Patent Application No. 60/710,276, filed Aug. 22, 2005, and I) claims priority to expired U.S. Provisional Patent Application No. 60/710,815, filed Aug. 24, 2005, the contents of each are incorporated herein by reference in their entireties; and

4) a Continuation-in-part of U.S. patent application Ser. No. 11/452,637, filed Jun. 14, 2006, which A) is a Continuation-in-Part of pending U.S. patent application Ser. No. 10/834,802, filed Apr. 29, 2004 (now U.S. Pat. No. 7,101,369, which issued Sep. 5, 2006), B) is a Continuation-in-Part of pending U.S. patent application Ser. No. 11/237,136, filed Sep. 28, 2005 which issued on Dec. 16, 2008 as U.S. Pat. No. 7,467,015, and which is a continuation-in-part of U.S. patent application Ser. No. 10/834,802, filed Apr. 29, 2004 which issued on Sep. 5, 2006 as U.S. Pat. No. 7,101,369, C) is a Continuation-in-Part of abandoned U.S. patent application Ser. No. 11/237,430, filed Sep. 28, 2005, and which is a continuation-in-part of U.S. patent application Ser. No. 10/834,802, filed Apr. 29, 2004 which issued on Sep. 5, 2006 as U.S. Pat. No. 7,101,369, D) is a Continuation-in-Part of U.S. patent application Ser. No. 11/236,985, filed Sep. 28, 2005, which issued on Jul. 17, 2007 as U.S. Pat. No. 7,244,254, and which is a continuation-in-part of U.S. patent application Ser. No. 10/834,802, filed Apr. 29, 2004 which issued on Sep. 5, 2006 as U.S. Pat. No. 7,101,369, E) is a Continuation-in-Part of abandoned U.S. patent application Ser. No. 11/440,331, filed May 24, 2006, which is a continuation-in-part of U.S. patent application Ser. No. 10/834,802, filed Apr. 29, 2004 which issued on Sep. 5, 2006 as U.S. Pat. No. 7,101,369, the contents of each are incorporated herein by reference in their entireties.

### BACKGROUND OF THE INVENTION

The present invention relates to medical instruments for ablating tissue, and in particular to a microwave probe for ablation of tumors and the like.

Microwave ablation (MWA), like radio frequency ablation (RFA), uses localized heating to cause tissue necrosis. However, MWA can produce greater and more rapid heating and can easily support the use of multiple probes because current flow between the probes can be limited. The mode of heating in MWA also eliminates ground pads and charring concerns.

Unfortunately, current MFA equipment produces relatively small lesions because of practical limits in power and treatment time. Power is limited by the current carrying capacity of the small gauge feeder line as it passes through the patient to the site of the necrosis. Larger feeder lines are undesirable because they are not easily inserted percutaneously. Heating of the feeder line at high powers can also lead to burns around the insertion point of the MWA probe.

Barrett's Esophagus is a precancerous condition of the esophagus that can progress to a type of cancer called esophageal adenocarcinoma. Barrett's esophagus is estimated to affect about 700,000 adults in the United States, and is associated with the very common condition gastroesophageal reflux disease or GERD. The risk of developing adenocarcinoma is 30 to 125 times higher in people who have Barrett's esophagus than in people who do not. While many people with Barrett's are asymptomatic and most will never progress to cancer, esophageal adenocarcinoma is often deadly as the condition is usually diagnosed late and the current treatments are not effective. Therefore, a treatment for Barrett's is needed that can effectively reduce the number of people that progress to adenocarcinoma without exposing asymptomatic people to unnecessary procedural complications and associated morbidity. The present disclosure fulfills this need.

Blood loss during surgery is a substantial clinical problem. Resection of multiple tissue types in the neck, chest, abdomen, pelvis, and extremities are associated with blood loss that can be acutely life-threatening from hemodynamic effects, or if the blood loss is severe enough, can require transfusions. This can be problematic from an immunological point of view during cancer surgery. For example, increased blood loss requiring transfusions during hepatic resection increases post-resection mortality. Blood loss is also a major problem during surgery for sharp or blunt trauma, in orthopedic surgery, and in gynecologic and obstetrical procedures.

Current electrosurgical devices used for cautery and cutting, discussed below, have various associated problems and disadvantages as are known in the art. Accordingly, there is a need for a device which decreases blood loss during surgery, which overcomes the problems and disadvantages associated with current electrosurgical devices used for cautery and cutting, and which is an improvement thereover.

Use of energy to ablate, resect or otherwise cause necrosis in diseased tissue has proven beneficial both to human and to animal health. Electrosurgery is a well-established technique to use electrical energy at DC or radio frequencies (i.e. less than 500 kHz) to simultaneously cut tissue and to coagulate small blood vessels. Radio-frequency (RF) ablation of tumor tissue was developed from the basis of electrosurgery, and has been used with varied success to coagulate blood vessels while creating zones of necrosis sufficient to kill tumor tissue with sufficient margin.

Limitations of the above techniques center on the need for ground pads on the skin of the patient to provide a return path for the current, as well as the undesirable stimulation of the nervous system as cuts are being made; this usually requires injection of a temporary paralyzing agent. Limitations of tissue impedance, particularly as the tissue becomes desiccated or charred during the course of the procedure, limit the amount of current, and hence the amount of ablative power, that can be applied to the tissue. This in turn limits the size of vessels that can be effectively shut down.

Thus current procedures are limited when applied to resection of tumors from highly-vascularized organs, e.g. liver. Furthermore, the limitations of current and power limit the speed at which these procedures can be performed. Accordingly, there is a need for a device which overcomes the problems and disadvantages associated with current procedures, and which is an improvement thereover. The present disclosure fulfills this need.

Varicose veins are a common medical condition that affect up to 60% of all Americans, and represent a significant health and cosmetic problem. Symptomatically, dilated varicose veins (usually the greater saphenous vein) can cause pain, cramping, itching, swelling, skin changes, venous stasis ulcers, and aching. The traditional therapy for treatment of varicose veins has been surgical removal (vein stripping), but currently less invasive treatments are becoming more common. Sclerotherapy (injection of a caustic substance to scar down the vein), laser and radiofrequency closure techniques, and minimally invasive surgery are becoming more popular. Energy delivery treatments (laser, radiofrequency, etc.) are promising because of their relatively low technical difficulty and good accuracy.

Limitations of the above techniques center on the means by which the vein in treated. Surgical techniques can be technically challenging and more invasive than energy delivery techniques or sclerotherapy. Sclerotherapy is limited in the accuracy by which substances may be administered. Laser techniques can cause the vein to become extremely hot, which increases the probability of burns to the skin and subcutaneous tissues as well as perforation of the vein. Radiofrequency techniques are relatively slow to heat, require ground pads to be placed on the patient and are not precise.

Accordingly, there is a need for a new and improved method and system to treat vascular pathologies such as varicose veins, which overcomes the above identified disadvantages and limitations of current vascular pathology and varicose vein treatment methods. The present disclosure fulfills this need.

### BRIEF SUMMARY OF THE INVENTION

The present invention provides a triaxial microwave probe design for MWA where the outer conductor allows improved tuning of the antenna to reduce reflected energy through the feeder line. This improved tuning reduces heating of the feeder line allowing more power to be applied to the tissue and/or a smaller feed line to be used. Further, the outer conductor may slide with respect to the inner conductors to permit adjustment of the tuning in vivo to correct for effects of the tissue on the tuning.

Specifically, the present invention provides a probe for microwave ablation having a first conductor and a tubular second conductor coaxially around the first conductor but insulated therefrom. A tubular third conductor is fit coaxially around the first and second conductors. The first conductor may extend beyond the second conductor into tissue when a proximal end of the probe is inserted into a body for microwave ablation. The second conductor may extend beyond the third conductor into the tissue to provide improved tuning of the probe limiting power dissipated in the probe outside of the exposed portions of the first and second conductors.

Thus, it is one object of at least one embodiment of the invention to provide improved tuning of an MWA device to provide greater power to a lesion without risking damage to the feed line or burning of tissue about the feed line and/or to allow smaller feed lines in microwave ablation.

The third tubular conductor may be a needle for insertion into the body. The needle may have a sharpened tip and may use an introducer to help insert it.

Thus, it is another object of at least one embodiment of the invention to provide a MWA probe that may make use of normal needle insertion techniques for placement of the probe.

It is another object of at least one embodiment of the invention to provide a rigid outer conductor that may support a standard coaxial for direct insertion into the body.

The first and second conductors may fit slidably within the third conductor.

It is another object of at least one embodiment of the invention to provide a probe that facilitates tuning of the probe in tissue by sliding the first and second conductors inside of a separate introducer needle.

The probe may include a lock attached to the third conductor to adjustably lock a sliding location of the first and second conductors with respect to the third conductor.

It is thus another object of at least one embodiment of the invention to allow locking of the probe once tuning is complete.

The probe may include a stop attached to the first and second conductors to about a second stop attached to the third conductor to set an amount the second conductor extends beyond the tubular third conductor into tissue. The stop may be adjustable.

Thus, it is another object of at least one embodiment of the invention to provide a method of rapidly setting the probe that allows for tuning after a coarse setting is obtained.

The second conductor may extend beyond the third conductor by an amount L 1 and the first conductor may extend beyond the second conductor by an amount L 2 and L 1 and L 2 may be multiples of a quarter wavelength of a microwave frequency received by the probe.

It is thus another object of at least one embodiment to promote a standing wave at an antenna portion of the probe.

These particular objects and advantages may apply to only some embodiments falling within the claims and thus do not define the scope of the invention.

This present disclosure relates to a microwave device that can be used for intraluminal tissue ablation, for example to effectively treat esophageal pathology, including (but not limited to) Barrett's Esophagus and esophageal adenocarcinoma. The preferred embodiment comprises a coaxial, triaxial or quadraxial microwave antenna housed in an esophageal dilator or balloon (FIG. 7). The proposed device can be introduced into the esophagus alongside or through an endoscope, and will deliver microwave energy to tissue. This energy heats the affected tissue, which subsequently undergoes necrosis thereby eliminating the potential of the tissue to undergo malignant transformation. The dilator or balloon is used to keep the antenna in the center of or approximate the center of the lumen allowing for generally symmetrical heating of the esophagus.

Other permutations of the preferred embodiment are possible. For example, the antenna need not be a triaxial antenna. Various microwave antennas could be used to heat the tissue, and other mechanisms of positioning the antenna in the center of the lumen are possible. It is also possible that heating elements could be incorporated into the dilator or balloon itself such that the heating occurs closer to the tissue. Similarly, the antenna(s) could be placed in close proximity to the tissue during the ablation (e.g. using a spiral shaped antenna) to treat the tissue.

This device is different than current devices that are used. For instance, this device will run in the microwave spectrum and receive power from a microwave generator rather than radiofrequency energy or lasers. The preferred frequencies would be 915 MHz and 2.45 GHz, but other frequencies could also be used. The depth of penetration of the coagulation effect can be varied depending on the amount of power that is applied, the location of the antenna relative to the tissue, and the duration of the power application (FIG. 5).

Accordingly, it is one of the objects of the present disclosure to provide a method and device for intralumenal tissue ablation.

It is another object of the present invention to provide a method and device to treat esophageal pathologies.

It is a further object of the present invention to provide a microwave device and method for intralumenal introduction and delivery of microwave energy to tissue.

Numerous other advantages and features of the disclosure will become readily apparent from the following detailed description, from the claims and from the accompanying drawings in which like numerals are employed to designate like parts throughout the same.

The device of the present disclosure is a microwave device that can be used to decrease blood loss during surgery. This device is different than electrocautery devices based on radiofrequency that are in widespread clinical use. The microwave surgical device described in this disclosure is comprised of a microwave antenna housed in a handset (or laparoscopic probe) that is placed in close proximity to the tissue of interest. When turned on (triggered), the device delivers microwave energy to tissue, providing a cautery or cutting, or combined cautery and cutting effect. Tissue can then be divided rapidly and without fear of untoward hemorrhage. This device can also be used to stop pre-existing hemorrhage on a small or large scale. For example, during open abdominal procedures, a small blood vessel can be near instantaneously cauterized by applying microwave energy directly to it.

Numerous other advantages and features of the disclosure will become readily apparent from the following detailed description, from the claims and from the accompanying drawings in which like numerals are employed to designate like parts throughout the same.

The present disclosure relates to delivery of microwave (e.g. approximately 800 MHz and higher frequencies) power to tissue for the purpose of ablating tissue or resecting tissue with little or no loss of blood.

The device enables delivery of large amounts of power (e.g. greater than 100 Watts) to tissue without the need for ground pads since it accomplishes an impedance match between tissue and the characteristic impedance of the waveguide that feeds power to it. This is accomplished in a hand-held format similar to many surgical tools. It can accept a variety of tips for different cutting and coagulation purposes. Furthermore, because of the impedance matching, reflected power from the tool is minimized. Reflected power can further be monitored at the generator or along the feed cable to use as feedback to the generator power control.

Numerous other advantages and features of the disclosure will become readily apparent from the following detailed description, from the claims and from the accompanying drawings in which like numerals are employed to designate like parts throughout the same.

The present disclosure relates to a method and system for vascular ablation using microwave energy to provide a very controllable heating pattern and to provide relatively fast heating, much faster for example than radiofrequency energy heating. The method and system delivers microwave (e.g. approximately 300 MHz and higher frequencies) power to a vessel wall, in particular for the treatment of vascular pathologies such as varicose veins.

The vascular ablation system generally comprises a microwave delivery device for heating the vessel wall, and a microwave power source for supplying microwave power to the delivery device. The vascular ablation system also preferably may include a cooling system, a temperature monitoring, feedback and control system, an ultrasound or other imaging device, and/or a device for assuring generally uniform energy delivery in the vein.

In a first embodiment, the microwave delivery device comprises a very thin microwave antenna that can be placed into the lumen of the vein. Focused microwave energy from an extracorporeal microwave power source would then be directed at this antenna transcutaneously to cause heating of the vessel wall and closure of the vein. Ferrite (or similar material) may be incorporated into the antenna wire to increase the heating effect of the external microwave field. Advantages of this approach include: (1) the intraluminal antenna could be very thin and minimally traumatic when placed inside the vein, (2) external heating could be primarily directed at the visible vessels on the leg surface, and (3) the external approach increases certainty of location of heat delivery, thus minimizing technical difficulty and reheating of already treated veins.

In a second embodiment, the microwave delivery device comprises a microwave antenna built into an endoluminal catheter that is specifically tuned to the impedance of the vessel wall. This tuning reduces reflected power, allowing the catheter to be very thin, reducing the trauma of antenna placement into the vein. The catheter could be a triaxial microwave catheter or other microwave antenna including center-fed dipole, dual-feed slot, segmented, or other microwave antennas. In this embodiment, the microwave power source comprises a co-axial cable for feeding microwave power to the antenna.

In a third embodiment, the microwave power source and the microwave delivery device are essentially integrated and comprise an external focused microwave source for heating of varicose veins that does not require an intracorporeal antenna. The superposition of microwave energy could be controlled transcutaneously to heat only the vessel walls desired. This microwave heating method is completely external and requires no invasiveness.

For transcutaneous heating, the microwave source could be attached to or used in conjunction with an ultrasound probe or other imaging devices or systems. With this method, the ultrasound probe could be used to localize the targeted vein in real-time. The vein could be compressed in any suitable manner to temporarily stop blood flow, and then sealed closed with focused microwave heating. Doppler ultrasound could then be used to confirm that the vein has no flow. Such a method could be used with or without an intracorporeal antenna.

With any of the embodiments described herein, a Mylar balloon (or an inflatable balloon or device of other conductive material) could be placed on the end of a catheter that is inserted into the vein. The balloon could be partially inflated to ensure that the catheter stays in contact with the vein wall to assure uniform energy delivery.

The vascular ablation system preferably may include a built-in cooling system to reduce skin burns when the microwave power source is external and placed on the skin. The cooling system may be separate or integrated into the microwave power source, such as a system of cooling channels, which may also be integrated into the ultrasound probe or other imaging device. The system can also provide for temperature monitoring at the skin surface.

The vascular ablation system preferably may include a temperature monitoring, feedback and control system used with any of the embodiments described herein. Temperature monitoring may be accomplished via a thermosensor in the catheter, and/or an external non-invasive temperature monitoring device.

The vascular ablation system may also include a method of compression, such as ultrasound guided compression or any other suitable compressing of the vessel, to stop blood flow and co-apt the vein walls during microwave ablation using any of the embodiments and methods described herein.

Accordingly, it is one of the objects of the present disclosure to provide a method and system for the controlled delivery of microwave power to a vessel wall such as a vein.

It is a further object of the present invention to provide a method and device for the delivery of microwave power to treat vascular pathologies such as varicose veins.

It is another object of the present invention to provide a method and system for vascular ablation.

Numerous other advantages and features of the disclosure will become readily apparent from the following detailed description, from the claims and from the accompanying drawings in which like numerals are employed to designate like parts throughout the same.

### BRIEF DESCRIPTION OF THE DRAWINGS

FIG. 1 is a schematic representation of a microwave power supply attached to a probe of the present invention for percutaneous delivery of microwave energy to a necrosis zone within an organ;

FIG. 2 is a perspective fragmentary view of the proximal end of the probe of FIG. 1 showing exposed portions of a first and second conductor slideably received by a third conductor and showing a sharpened introducer used for placement of the third conductor;

FIG. 3 is a fragmentary cross sectional view of the probe of FIG. 2 showing connection of the microwave power supply to the first and second conductors; and

FIG. 4 is a cross sectional view of an alternative embodiment of the probe showing a distal electric connector plus an adjustable stop thumb screw and lock for tuning the probe;

FIG. 5 is a chart illustrating the performance of the device of the preferred embodiment of the present disclosure. In addition, FIG. 5 shows dependence of microwave coagulation diameter in liver on a) time and b) applied power; note that increasing either parameter results in an increased coagulation diameter.

FIG. 6 is a schematic diagram of the device of the preferred embodiment of the present disclosure in use in an esophagus using the balloon (left), and using the dilator (right).

FIG. 7A is a chart illustrating the dependence of the coagulation diameter on the length of time of use of the device of the present disclosure.

FIG. 7B is a chart illustrating the dependence of the coagulation diameter on the amount of applied power during use of the device of the present disclosure.

FIG. 8 is a diagram of a delivery tool and control/feedback system for cauterizing tissue, illustrating a preferred embodiment of the present disclosure.

FIG. 9 is a schematic, cross-sectional diagram of an embodiment of an antenna and scalpel combination of the present disclosure.

FIG. 10 is a schematic diagram of an embodiment of an antenna and scissors combination of the present disclosure.

FIG. 11 is a cross-sectional view of a preferred embodiment of the present disclosure, showing the arrangement of an impedance-matching sleeve and the tip.

FIG. 12 is a plan view of the preferred embodiment of the present disclosure encapsulated in a ceramic or plastic housing.

FIG. 13 is a schematic circuit diagram for a microwave power delivery and control system in accordance with the preferred embodiment of the present disclosure.

FIG. 14 is a schematic cross-sectional view of a first embodiment of the present invention, showing the antenna and microwave source relative to a vessel.

FIG. 15 is a schematic cross-sectional view of a second embodiment of the present invention, showing a radiating microwave antenna placed inside the vessel.

FIG. 16 is a schematic cross-sectional view of a third embodiment of the present invention, showing an integrated external microwave source and delivery device focused on an area inside the vessel.

FIG. 17 is a schematic cross-sectional view of an alternate embodiment of the present invention, showing a balloon used to maintain the position of an antenna relative to the vessel walls.

### DETAILED DESCRIPTION OF EMBODIMENTS

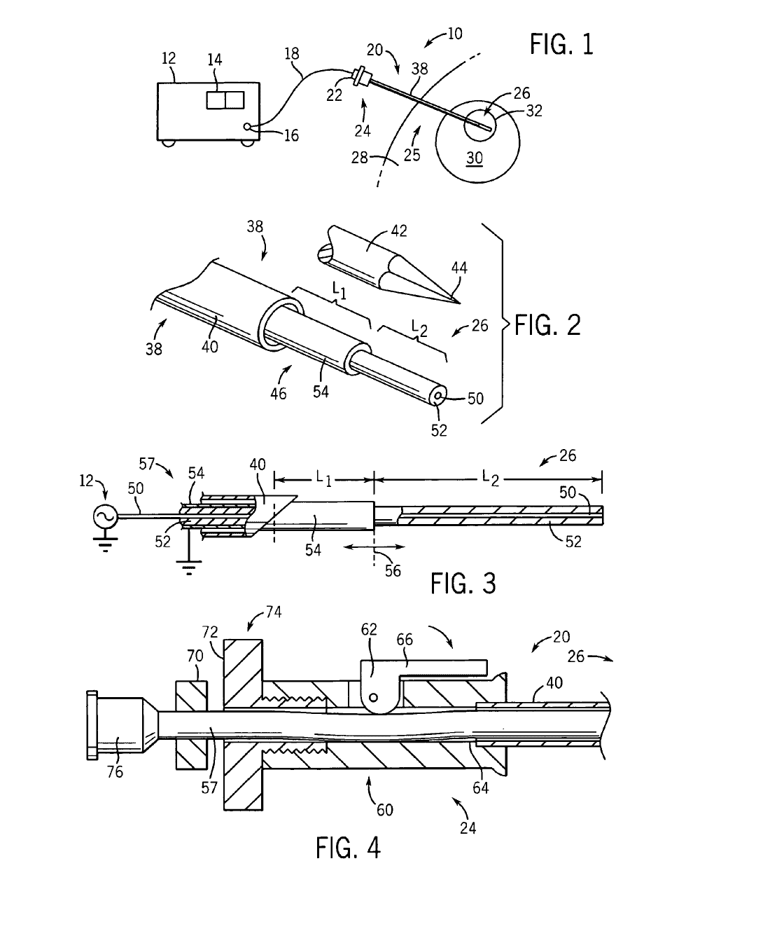

Referring now to FIG. 1, a microwave ablation device 10 per the present invention includes a microwave power supply 12 having an output jack 16 connected to a flexible coaxial cable 18 of a type well known in the art. The cable 18 may in turn connect to a probe 20 via a connector 22 at a distal end 24 of the probe 20.

The probe 20 provides a shaft 38 supporting at a proximal end 25 an antenna portion 26 which may be inserted percutaneously into a patient 28 to an ablation site 32 in an organ 30 such as the liver or the like.

The microwave power supply 12 may provide a standing wave or reflected power meter 14 or the like and in the preferred embodiment may provide as much as 100 watts of microwave power of a frequency of 2.45 GHz. Such microwave power supplies are available from a wide variety of commercial sources including as Cober-Muegge, LLC of Norwalk, Conn., USA.

Referring now to FIGS. 1 and 2, generally a shaft 38 of the probe 20 includes an electrically conductive tubular needle 40 being, for example, an 18-gauge needle of suitable length to penetrate the patient 28 to the ablation site 32 maintaining a distal end 24 outside of the patient 28 for manipulation.

Either an introducer 42 or a coaxial conductor 46 may fit within the needle 40. The introducer 42 may be a sharpened rod of a type well known in the art that plugs the opening of the needle 40 and provides a point 44 facilitating the insertion of the probe 20 through tissue to the ablation site 32. The needle 40 and introducer 42 are of rigid material, for example, stainless steel, providing strength and allowing easy imaging using ultrasound or the like.

The coaxial conductor 46 providing a central first conductor 50 surrounded by an insulating dielectric layer 52 in turn surrounded by a second outer coaxial shield 54. This outer shield 54 may be surrounded by an outer insulating dielectric not shown in FIG. 2 or may be received directly into the needle 40 with only an insulating air gap between the two. The coaxial conductor 46 may, for example, be a low loss 0.86-millimeter coaxial cable.

Referring still to FIG. 2, the central conductor 50 with or without the dielectric layer 52, extends a distance L 2 out from the conductor of the shield 54 whereas the shield 54 extends a distance L 1 out from the conductor of the needle 40. L 1 is adjusted to be an odd multiple of one quarter of the wavelength of the frequency of the microwave energy from the power supply 12. Thus the central conductor 50 in the region of L 2 provides a resonant monopole antenna having a peak electrical field at its proximal end and a minimal electric field at the end of the shield 54 as indicated by 56.

At 2.45 GHz, the length L 2 could be as little as 4.66 millimeters. Preferably, however, a higher multiple is used, for example, three times the quarter wavelength of the microwave power making L 2 approximately fourteen millimeters in length. This length may be further increased by multiple half wavelengths, if needed.

Referring to FIG. 3, the length L 1 is also selected to be an odd multiple of one quarter of the wavelength of the frequency of the microwave energy from the power supply 12. When needle 40 has a sharpened or bevel cut tip, distance L 1 is the average distance along the axis of the needle 40 of the tip of needle 40.

The purpose of L 1 is to enforce a zero electrical field boundary condition at line 56 and to match the feeder line 56 being a continuation of coaxial conductor 46 within the needle 40 to that of the antenna portion 26. This significantly reduces reflected energy from the antenna portion 26 into the feeder line 56 preventing the formation of standing waves which can create hot spots of high current. In the preferred embodiment, L 1 equals L 2 which is approximately fourteen millimeters.

The inventors have determined that the needle 40 need not be electrically connected to the power supply 12 or to the shield 54 other than by capacitive or inductive coupling. On the other hand, small amounts of ohmic contact between shield 54 and needle 40 may be tolerated.

Referring now to FIGS. 1, 2 and 4, during use, the combination of the needle 40 and introducer 42 are inserted into the patient 28, and then the introducer 42 is withdrawn and replaced by a the coaxial conductor 46 so that the distance L 2 is roughly established. L 2 has been previously empirically for typical tissue by trimming the conductor 50 as necessary.

The distal end 24 of needle 40 may include a tuning mechanism 60 attached to the needle 40 and providing an inner channel 64 aligned with the lumen of the needle 40. The tuning mechanism provides at its distal end, a thumbwheel 72 having a threaded portion received by corresponding threads in a housing of the tuning mechanism and an outer knurled surface 74. A distal face of the thumbwheel provides a stop that may abut a second stop 70 being clamped to the coaxial conductor 46 thread through the tuning mechanism 60 and needle 40. When the stops 70 and on thumbwheel 72 about each other, the coaxial conductor 46 will be approximately at the right location to provide for extension L 1. Rotation of the thumbwheel 72 allows further retraction of the coaxial conductor 46 to bring the probe 20 into tuning by adjusting L 1. The tuning may be assessed by observing the reflected power meter 14 of FIG. 1 and tuning for reduced reflected energy.

The tuning mechanism 60 further provides a cam 62 adjacent to the inner channel 64 through which the coaxial conductor 46 may pass so that the cam 62 may press and hold the coaxial conductor 46 against the inner surface of the channel 64 when a cam lever 66 is pressed downwards 68. Thus, once L 1 is properly tuned, the coaxial conductor 46 may be locked in position with respect to needle 40.

The distal end of the coaxial conductor 46 may be attached to an electrical connector 76 allowing the cable 18 to be removably attached to disposable probes 20.

The present invention provides as much as a ten-decibel decrease in reflected energy over a simple coaxial monopole in simulation experiments and can create a region of necrosis at the ablation site 32 greater than two centimeters in diameter.

It is specifically intended that the present invention not be limited to the embodiments and illustrations contained herein, but include modified forms of those embodiments including portions of the embodiments and combinations of elements of different embodiments as come within the scope of the following claims.

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

This present disclosure illustrates a microwave device that can be used to effectively treat esophageal pathology, including (but not limited to) Barrett's Esophagus and esophageal adenocarcinoma. The preferred embodiment comprises a coaxial, triaxial or quadraxial microwave antenna, which is housed in either an esophageal dilator or a dilator balloon (e.g., between the dilator and the antenna) when in use. In some embodiments, the antenna is separate from the dilator and the balloon.

This device is different than current devices that are used. For instance, this device will run in the microwave spectrum and receive power from a microwave generator rather than radiofrequency energy or lasers. The preferred frequencies would be 915 MHz and 2.45 GHz, but other frequencies could also be used.

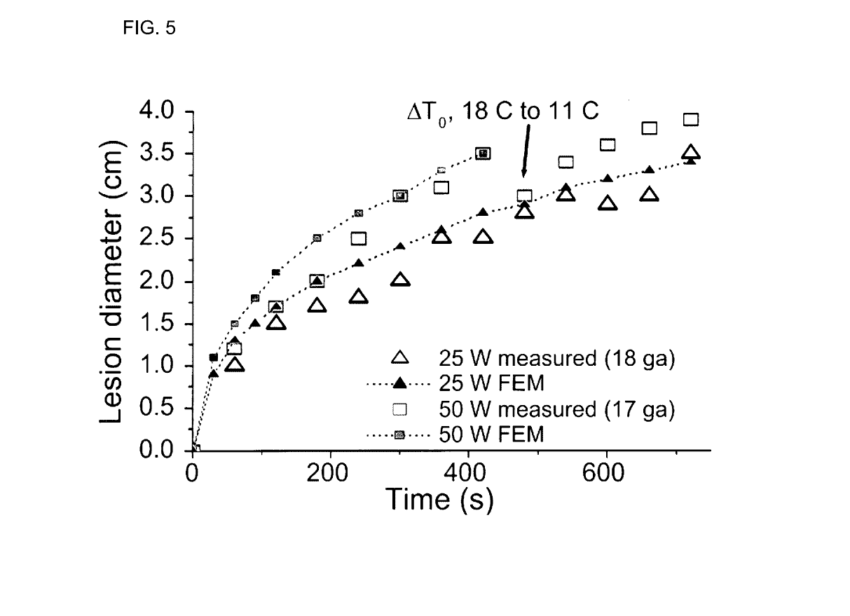

As illustrated in FIG. 5, the depth of penetration of the coagulation effect can be varied depending on the amount of power that is applied, the location of the antenna relative to the tissue, and the duration of the power application. The chart in FIG. 5 illustrates the dependence of microwave coagulation diameter or lesion diameter in liver on a) time and b) applied power. It is noted that increasing either parameter (time or power) results in an increased coagulation diameter.

The proposed device can be introduced into the esophagus alongside or through an endoscope, and will deliver microwave energy to tissue. This energy heats the affected tissue, which subsequently undergoes necrosis thereby eliminating the potential of the tissue to undergo malignant transformation.

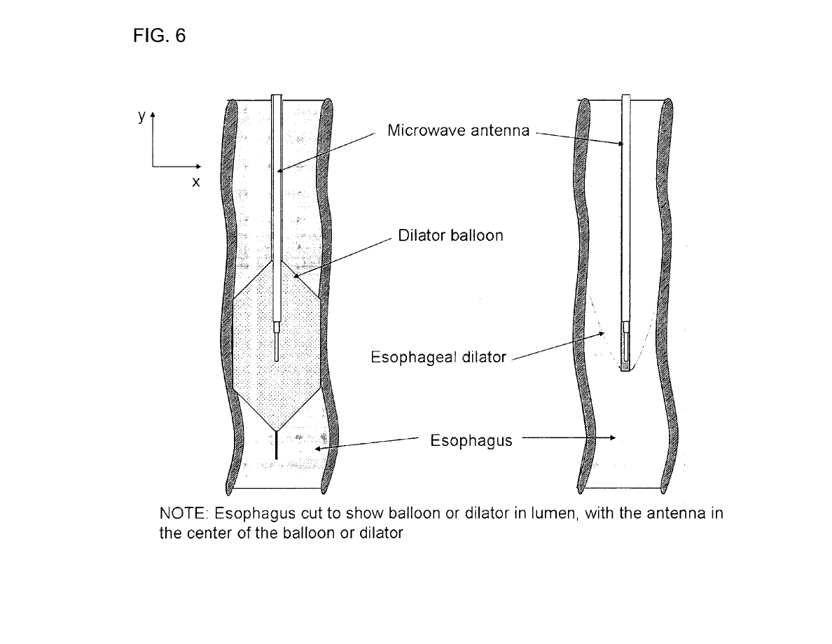

As can be seen in FIG. 6, the esophageal dilator (right side diagram) or the dilator balloon (left side diagram) is used to keep the antenna in the center of or proximate the center of the lumen allowing for generally symmetrical heating of the esophagus. Microwave energy can be fed to the antenna with a coaxial transmission line or dielectric or hollow-pipe waveguide. The applicator beneficially does not require conductive contact to the tissue under treatment.

It should be understood based upon the present disclosure that other permutations or modifications of the preferred embodiment are possible. For example, the antenna need not be a triaxial antenna. Various microwave antennas could be used to heat the tissue, and other mechanisms of positioning the antenna in the center of the lumen are possible and contemplated. It is also possible that heating elements could be incorporated directly into the dilator or balloon itself such that the heating occurs closer to the tissue. Similarly, the antenna(s) could be placed in close proximity to the tissue during the ablation (e.g. using a spiral shaped antenna) to treat the tissue. In general, any suitable power supply and microwave applicator combination for treatment of esophageal pathologies or other pathologies that can be introduced into a lumen through a breathing tube, a balloon dilator or any other like device is contemplated.

It is to be understood that the embodiment(s) herein described is/are merely illustrative of the principles of the present invention. Various modifications may be made by those skilled in the art without departing from the spirit or scope of the claims which follow.

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

The device of the present disclosure is different than current electrosurgical devices that are used for cautery and cutting. The disclosed device will run in the microwave (not radiofrequency) spectrum and receives power from a from a microwave generator. The preferred frequencies would be the ISM (Industrial, Scientific and Medical) bands at 915 MHz, 2.45 GHz, and 5.8 GHz, although other frequencies could also be used. Since the device is not radiofrequency based, there is no need for ground pads, and charring will not substantially affect the ability of this device to perform a cautery or cut function.

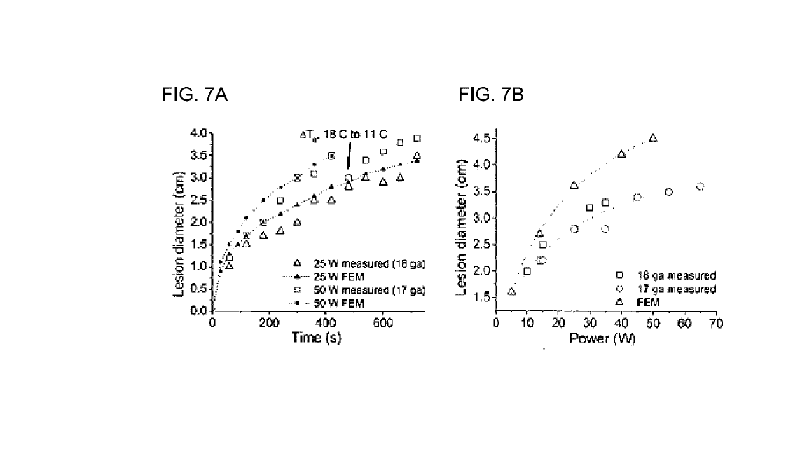

The depth of penetration of the coagulation effect can be varied depending on the amount of power that is applied, the angle at which the device is held, and the duration that the device is held in proximity to the tissue. For example, experimental data show that a region greater than 2 cm in diameter can be coagulated in 2 minutes with an input power of ^{˜}65 W. Data also shows the ablation zone diameter may be controlled by varying input power and application time (FIGS. 7A and 7B).

The specific antenna design can be variable. One possibility is to construct the microwave delivery tool based on a triaxial design, thereby taking advantage of the resonant frequency effects of triaxial catheters. However, many microwave delivery systems (e.g. coaxial near-field antennas) can be used for this purpose if they are designed to have a short protrusion of the center conductor (e.g. protrusion approximately the radius of the coaxial cable) such that in near-contact with tissue, a large absorption of microwave power is achieved.

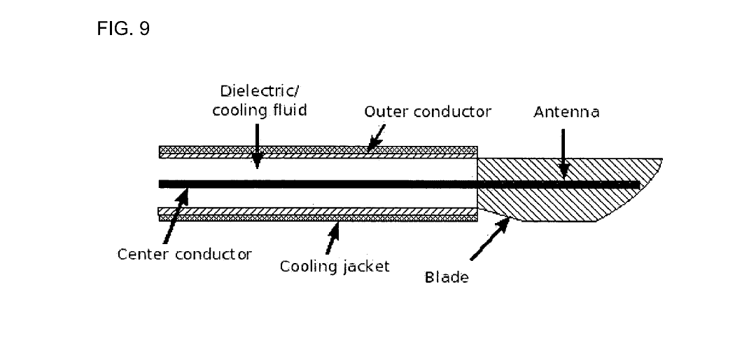

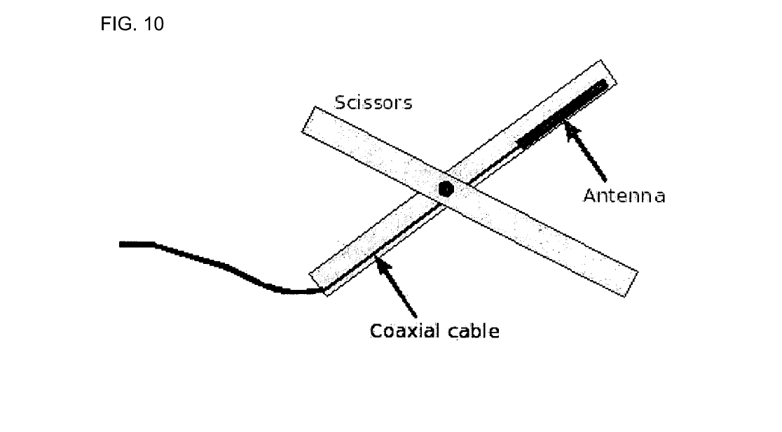

Other antenna designs may include dielectric resonators, particularly those formed in the shape of a mechanical cutting tool; coplanar, microstrip or similar waveguiding and radiating structures; spiral or helical antennas with the helix axis parallel to the coaxial feed line; planar spiral antennas; two-sided balanced or unbalanced transmission lines; antennas mounted as part of a scissors (FIG. 10), knife or scalpel (FIG. 9), clamp or other cutting or pressure-inducing device.

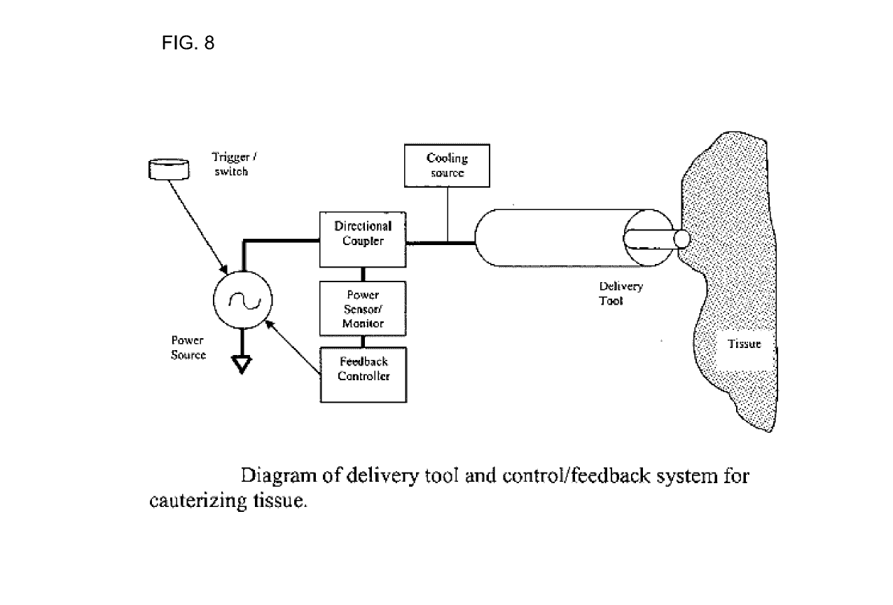

As shown in FIG. 8, the system may deliver power to the tool through a trigger switch, foot pedal or other switch or on/off button. Power reflected from the antenna can be detected and monitored to provide feedback for power control or as a safety interlock to interrupt the microwave power source if the reflected power exceeds a threshold. The control and feedback loop varies the power or duty cycle of the microwave source, enabling both safe operation and variable power application. Further, the tool can have an adjustment or calibration mechanism wherein the device can be tuned relative to the tissue of interest to a low reflected power prior to use.

The device can be mounted in a handle that is cooled by circulating fluid, gas or liquid metal. In addition, cooling fluid, gas, or liquid metal can be circulated through the center of the antenna to reduce untoward line heating as well as vary the characteristic impedance of the antenna. In one embodiment, the antenna operates at a preferential frequency of 77Ω to reduce line heating. Alternatively or in addition, the antenna can have an air-core or vacuum-core design to reduce dielectric heating. The feed of the antenna can be comprised of any conductive metal including copper, stainless steel or titanium, and the shaft can be insulated with various thermal insulators such as parylene or Teflon. The delivery tool can be coated with a biocompatible coating (e.g. a polymer such as Paralyne), and can be cooled with a water jacket.

As stated previously, this device could be used at conventional open surgery, laparoscopy, and/or percutaneously for the purpose of coagulation, vessel sealing, or cutting. The application end could house a mechanical scalpel or any other type of device to divide tissue to make an “all in one” coagulation and cutting device. The antenna could be mounted in combination with other surgical tools (one example is with a conventional scalpel), scissors, or used as a needle to stop hemorrhage. The depth of electromagnetic field penetration could be varied depending on the particular use; for example in neurosurgery, a very small amount of penetration would be desirable.

It is to be understood that the embodiment(s) herein described is/are merely illustrative of the principles of the present invention. Various modifications may be made by those skilled in the art without departing from the spirit or scope of the claims which follow.

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

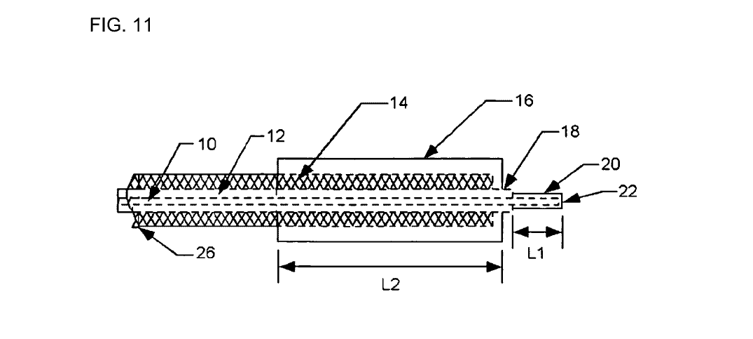

With reference to the drawings, an example of the preferred embodiment of the energy delivery device or microwave tissue resection tool of the present disclosure is shown in FIG. 11.

As illustrated in FIG. 11, a semi-rigid coaxial cable, preferably constructed of copper or silver with a suitable low-loss dielectric, forms the basis of the device. The cable's center conductor 10 protrudes from the outer conductor 12 by a length L 1, which is set to be a λ/4 (quarter-wavelength) at the frequency of excitation (e.g. 915 MHz, 2.45 GHz, or another suitable frequency) in the dielectric environment of the tissue of interest. The cable can be chosen from commercially-available standards, but it should be thick enough to be rated for the power delivered.

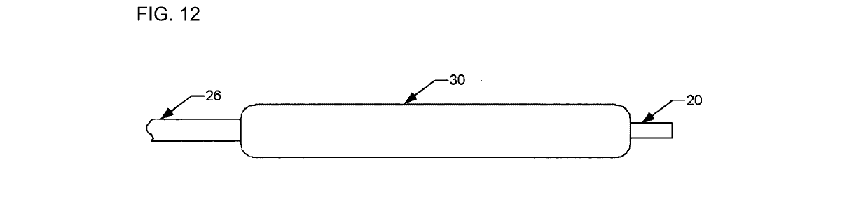

The coaxial cable is shrouded by a dielectric sleeve 14 that provides both thermal and electrical insulation. Fitted against this sleeve is a conductive sleeve (e.g. made of copper or silver or another suitable conductor) whose length is set to be a λ/4 (quarter-wavelength) at the frequency of excitation (e.g. 915 MHz, 2.45 GHz, or another suitable frequency) in the dielectric environment of the dielectric sleeve 14 and the shroud 30 (FIG. 12). This conductive sleeve 16 contacts the outer conductor of the coaxial cable 12 at a point 18, where it is free to slide if necessary to fine-tune the impedance matching effect. It can then be fixed in place with adhesive or other suitable mechanism.

The protrusion of the coaxial cable's center conductor 10 is shrouded by a non-stick material 20 (e.g. PTFE or Teflon) to minimize adhesion of the device to the tissue. A tip 22 at the distal end of the device can be specially formed to maximize the electric field emanating from it. For example, the tip 22 can be sharpened and optionally exposed directly to the tissue.

The device is connected to a feed cable at its proximal end 26. This cable can be optionally connectorized, by attaching any suitable connector known in the art of connecting cable, to simplify exchange of the device.

As shown in FIG. 12, the device can be enshrouded in a suitable ceramic or plastic housing 30, which can contain cooling fluid (e.g. air, nitrogen, water, etc) and microwave absorbing material (e.g. polyiron) to minimize radiation from the tool to the extent necessary or desired.

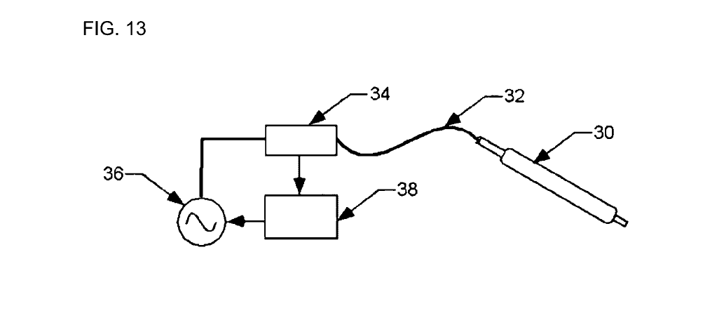

As shown in FIG. 13, the device 30 can be used in a system by which it is connected to a source of microwave power 36 via a cable 32. A directional coupler or other wave-sampling mechanism 34 in combination with a power sensor and feedback circuit 38 can be used to monitor reflected power from the device during the procedure. If the amount of reflected power exceeds a threshold, power from the generator 36 can be reduced to minimize heating of the device 30, while if the amount of reflected power is below a threshold, power can be increased to speed the procedure.

It is to be understood that the embodiment(s) herein described is/are merely illustrative of the principles of the present invention. Various modifications may be made by those skilled in the art without departing from the spirit or scope of the claims which follow.

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

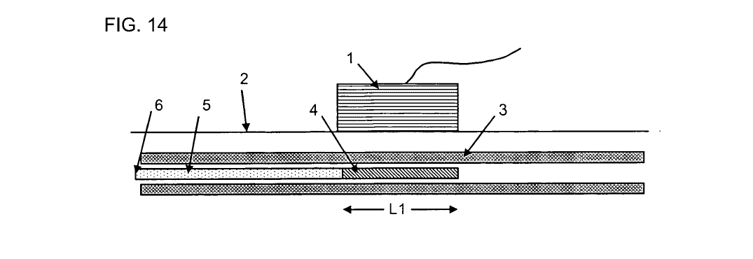

FIGS. 14-16 illustrate several embodiments of the vascular ablation method and system of the present disclosure is shown.

As illustrated in FIG. 14, a first embodiment of the present disclosure comprises a thin metallic wire antenna 4 positioned inside the vessel 3 by a non-radiating catheter 5. The antenna 4 may be purely metallic or contain a core or sections of ferrite or similar material to enhance the heating effect. For small, tortuous veins, the antenna/catheter should be flexible enough to migrate therethrough. An external microwave source 1 positioned proximate the skin surface 2 directs energy at the wire antenna 4 causing the antenna 4 to radiate locally, thereby focusing the microwave energy on the wall of the vessel 3 to heat and ablate the vessel 3. The length L 1 of the antenna 4 is arbitrary. The placement catheter 5 is located at the proximal end 6.

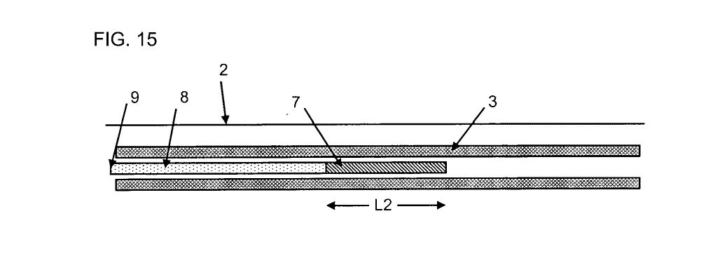

As illustrated in FIG. 15, a second embodiment of the present disclosure comprises a coaxial cable 9 which feeds the radiating antenna 7 directly with microwave energy. That energy is radiated by the antenna 7 to the wall of the vessel 3. The antenna length L 2 is fixed by the frequency of the microwave energy applied.

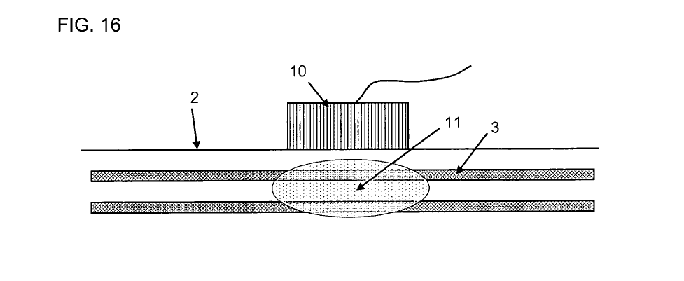

As illustrated in FIG. 16, a third embodiment of the present disclosure comprises an external microwave source 10 controlled in such a way as to focus radiated energy in a small volume 11 onto the vessel 3. The energy is applied transcutaneously.

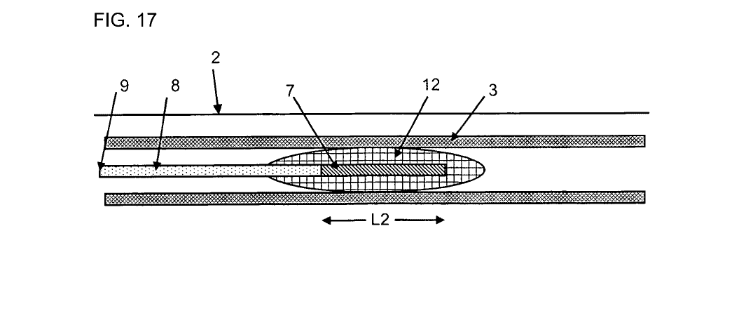

In any of the three embodiments described above, a device such as a balloon may be used to assist in providing generally uniform energy delivery in the vessel. As illustrated in FIG. 17, the balloon 12, comprised of conductive material such as Mylar, is shown in use in the vessel 3 to hold the position of the antenna 7 relative to the vessel wall.

Further, the vascular method and system of the present disclosure may include the use of an ultrasound probe or other imaging system or device to guide the antennas into place in the vessels. The ultrasound probe may also house the microwave source, such as the external microwave source 1 shown in FIG. 14, or external microwave source 10 shown in FIG. 16. The ultrasound probe and/or the external microwave source 1 or 10, may also house a cooling system to be placed on the skin 2 to cool the skin. The ultrasound probe may also be used to compress the skin 2 and vessel 3 during use of any energy delivery system to stop blood flow and allow full treatment of the vessel wall. It should be understood that the vessel may be compressed in any suitable manner, and the use of the ultrasound probe is just one example of such compression.

Still further, a thermosensor or external thermometry system may be used to measure the temperature of the vessel wall and/or the skin surface and provide feedback. Temperature information may be used in a feedback loop to control the microwave power applied, location of focused heating, antenna placement or treatment duration.

It is to be understood that the embodiment(s) herein described is/are merely illustrative of the principles of the present invention. Various modifications may be made by those skilled in the art without departing from the spirit or scope of the claims which follow. For example, the antenna/catheter may include an LED or other indicator that can be observed through the skin or otherwise used to monitor position of the antenna, especially near a patient's saphenofemoral junction. Further, the antenna can be coated with any suitable material or coating to prevent the antenna from adhering to the clot forming in the vein and/or to the vein wall during use.

With respect to the delivery of energy to the vein, the embodiments disclosed herein may include both pulsed and continuous energy delivery. A foot pedal or any other suitable switch or trigger device may be incorporated to allow the user to selectively switch energy delivery on/off. Microwave ablation of veins may be achieved using continuous power application, or by sequentially treating segments of the vein and pulling the antenna back between each. Different power schedules/powers for large (e.g. >5 mm) and small veins can be used or delivered. Also, multiple external power sources with destructive/constructive interference capability may be incorporated and used in the disclosed embodiments. Any combination of external power sources are contemplated, not just microwave, but also, for example, high-frequency ultrasound (hiFU), radio frequency (RF), and any other suitable external power sources. Further, compression of the vessel can be used with any external power source(s) or combinations thereof.

Additionally, the embodiments disclosed herein may be used in combination with any imaging monitoring (CT, US, MRI, fluoroscopy, mammography, nuclear medicine, etc.). With respect to the use of ultrasound, the antenna/catheter may have an echogenic coating or surface for better US visualization. Feedback systems (temperature, doppler, reflected power, etc.) and audio or visual indicators may be incorporated and used to advise the user or operator to hold/change the current position or retraction rate. The disclosed embodiments can incorporate and use software for targeting (in combination with imaging guidance), similar to a biopsy guide with ultrasound. This could assure that all of the power sources are focused on the same target.

We claim:

1. A probe for microwave ablation comprising: a first conductor; a tubular second conductor coaxially around the first conductor but insulated therefrom; a tubular third conductor coaxially around the first and second conductors; a tuning mechanism having a locked state fixedly holding the third conductor against axial movement with respect to the first and second conductors and having a unlocked state allowing axial movement between the third conductor and the first and second conductors wherein the first conductor extends beyond the second conductor into tissue, when a distal end of the probe is inserted into a body for microwave ablation, to promote microwave frequency current flow between the first and second conductors through the tissue; and wherein the second conductor may be adjusted by the tuning mechanism to extend beyond the third conductor into tissue when an end of the probe is inserted into the body for microwave ablation to provide improved tuning of the probe limiting power dissipated in the probe outside of exposed portions of the first and second conductors.
